(19) United States
(12) Patent Application Publication
(10) Pub. No.: US 2021/0346715 A1
(43) Pub. Date: Nov. 11, 2021

(54) Light Energy Therapy for Acute Respiratory Distress Syndrome

(71) Applicant: Erchonia Corporation LLC, Melbourne, FL (US)

(72) Inventors: Steven C SHANKS, Melbourne, FL (US); Rodrigo NEIRA, Alberta (CA); Rod NEIRA, Alberta (CA); Clara NEIRA, Melbourne, FL (US); Travis SAMMONS, Melbourne, FL (US)

(73) Assignee: Erchonia Corporation, LLC, Melbourne, FL (US)

(21) Appl. No.: 17/232,535

(22) Filed: Apr. 16, 2021

Related U.S. Application Data
(60) Provisional application No. 63/012,093, filed on Apr. 18, 2020.

(51) Int. Cl. A61N 5/06; A61N 5/067

(52) U.S. Cl. CPC A61N 5/0613; A61N 2005/0626; A61N 2005/0663; A61N 5/067

(57) ABSTRACT

Methods for treating acute respiratory distress syndrome are disclosed. Light energy is applied externally to a patient at or near targeted areas including the lungs, the vagus nerve, and spinous process to stimulate different neurological pathways and reduce inflammation. The wavelength of the applied light ranges from about 400-700 nm, and in a preferred embodiment uses two wavelengths, 405 nm and 635 nm. The applied light energy can be applied in pulses or as a constant wave. The light energy is applied at dosages that cause no detectable temperature rise of the treated tissue and no macroscopically visible changes in tissue structure. Preferably the power is 500 mW or less. The light can be emitted from the same light emitter or from multiple emitters and is preferably laser light.

### CROSS-REFERENCE TO RELATED APPLICATIONS

This application claims the benefit of co-pending U.S. Provisional Application No. 63/012,093 filed Apr. 18, 2020.

### FIELD OF INVENTION

This invention relates generally to methods for treating acute respiratory distress syndrome. This invention relates more particularly to applying light energy externally to a patient at certain areas of the body to reduce inflammation, reduce fibrosis in the lungs, and boost the immune function to treat acute respiratory distress syndrome.

### BACKGROUND

Acute respiratory distress syndrome (ARDS) occurs when fluid builds up in the alveoli of a patient's lungs. The fluid in these tiny elastic air sacs keeps the lungs from filling with enough air, which means less oxygen reaches the bloodstream. This in turn deprives the patient's organs of sufficient oxygen to function. ARDS typically occurs in people who are already critically ill or who have significant injuries; it may be contracted due to coronavirus, other virus, trauma, or sepsis. There is currently no known treatment for ARDS.

ARDS manifests as severe shortness of breath and may be accompanied by high fever, body aches, or cough for some diseases such as COVID-19. Some people develop mild symptoms while other patients develop severe symptoms and, even when put on a ventilator as a last resort, succumb and die. The risk of death increases with age and severity of illness. Of the people who do survive ARDS, some recover completely while others experience lasting damage to their lungs and other organs. Patients with ARDS often develop scar tissue in their lungs and will have long-term consequences if the fibrosis is not treated.

For the sickest patients, infection with the new coronavirus, named severe acute respiratory syndrome coronavirus 2 or SARS-CoV-2, is proving to be a full-body assault, causing damage well beyond the lungs. And even after patients who become severely ill have recovered and cleared the virus, physicians have begun seeing evidence of the infection's lingering effects. SARS-CoV-2, also known as COVID-19, can affect the heart, the liver, the kidneys, the brain, the endocrine system and the blood system. Medical experts are predicting long-term downstream effects of the coronavirus infection, akin to those seen with other viral infections such as herpes and Ebolavirus.

The immune system reacts to ARDS by creating an inflammatory pathway that if not treated can eventually kill the patient. Local inflammatory cells (neutrophil granulocytes and macrophages) secrete a number of cytokines into the bloodstream, most notable of which are the interleukins IL1 and IL6 and TNFα. The liver responds by producing many acute-phase reactants. The terms acute-phase protein and acute-phase reactant are often used synonymously. Acute-phase proteins are a class of proteins whose plasma concentrations increase (positive acute-phase proteins) or decrease (negative acute-phase proteins) in response to inflammation. This response is called the acute-phase reaction. The acute-phase reaction characteristically involves fever, acceleration of peripheral leukocytes, circulating neutrophils and their precursors. Increased acute-phase proteins from the liver may also contribute to the promotion of sepsis. At the same time, the production of a number of other proteins is reduced. These proteins are therefore referred to as “negative” acute-phase reactants.

The latest research also has shown that the digestive system can play a key role in ARDS.

It would be advantageous to treat ARDS by reducing inflammation, improving the function of the lungs and gut, and boosting the body's immune function.

### SUMMARY OF THE INVENTION

The present invention is a non-invasive method for treating acute respiratory distress syndrome. Certain areas of the patient's body are treated with light energy to combat the onslaught of underlying mechanisms that the immune system is reacting to causing ARDS to manifest itself as labored breathing, high fever, body aches, or cough. This invention involves applying light energy externally to a patient at targeted areas to reduce inflammation, reduce fibrosis in the lungs, improve the function of the digestive tract, and boost the immune function by treating the neurological system.

Light energy is applied externally to a patient at or near targeted areas including at or near the lungs, the vagus nerve, and spinous process, to stimulate different neurological pathways and reduce inflammation. The wavelength of the applied light ranges from about 400-700 nm, and in a preferred embodiment uses two wavelengths, 405 nm and 635 nm. The applied light energy can be applied in pulses or as a constant wave. The light energy is applied at dosages that cause no detectable temperature rise of the treated tissue and no macroscopically visible changes in tissue structure. Preferably the power is 500 mW or less. The light can be emitted from the same light emitter or from multiple emitters and is preferably laser light.

### BRIEF DESCRIPTION OF THE DRAWINGS

FIG. 1 illustrates light energy being applied to a patient's lungs using a hand-held device.

FIG. 2 illustrates light energy being applied to a patient's spinous process using a hand-held device.

FIG. 3 illustrates light energy being applied to a patient's vagus nerve on the right side of a patient's neck.

### DETAILED DESCRIPTION OF THE INVENTION

This invention treats ARDS by applying light energy externally to a patient at certain areas to reduce inflammation, reduce fibrosis in the lungs, and boost the immune function by treating the neurological system.

Low-level laser therapy (“LLLT”) has been shown through numerous clinical studies and regulatory clearances to be a safe, effective, simple, non-invasive and side-effect free alternative to medication and surgical procedures in a variety of situations. The light energy is applied at dosages that cause no detectable temperature rise of the treated tissue and no macroscopically visible changes in tissue structure. Consequently, the tissue impinged by the light is not heated and not damaged. LLLT is also known as photobiomodulation because its effect is photochemical not thermal. LLLT applied at proper dosages and locations reduces edema, improves wound healing, and relieves pain of various etiologies. It is also used in the treatment and repair of injured muscles and tendons.

Application of LLLT has been shown to have the potential to alter cellular metabolism to produce a beneficial clinical effect. For example, when hypoxic or otherwise impaired cells are irradiated with LLLT, mitochondrial adenosine tri-phosphate (ATP) production increases and nitric oxide is released. When exposed to near-infrared photons, cytochrome-C oxidase (CCO) releases nitric oxide which diffuses out the cell, increasing local blood flow and vasodilation, effecting a brief burst of reactive oxygen species (ROS) in the neuron cell. This in turn activates numerous signaling pathways that activate redox-sensitive genes and related transcription factors.

Light therapy is used to treat inflammation. Tumor necrosis factor alpha (TNFα) is a cell-signaling protein (cytokine) involved in systemic inflammation and is one of the cytokines that make up the acute phase reaction. The primary role of TNF is in the regulation of the body's leukocytes, also known as white blood cells. The elimination or reduction of the inflammation will result in milder systems and will help prevent the ARDS patient from having to be put on a ventilator, the treatment of last resort in the COVID-19 pandemic. Light therapy has been shown to reduce TNFα at 635 nm wavelength and thus reduce inflammation.

Light therapy is used to treat fibrosis in the lungs. Patients with ARDS also develop scar tissue in their lungs and will have long term consequences if the fibrosis is not treated. Light energy from 405-650 nm has been proven to reduce fibrosis in the tissue, with 405 nm proven the most effective.

The latest research also has shown that the digestive system can play a key role in ARDS and light therapy is used to treat the immune response from the digestive track. The vagus nerve is the gut-brain access. As used herein, gut means the gastrointestinal system, also referred to as the gastrointestinal tract, digestive system, digestive tract, or gut, and is a group of organs that includes the mouth, esophagus, stomach, pancreas, liver, gallbladder, small intestine, colon, and rectum. Vagus nerve refers to either of the 10th pair of cranial nerves that arise from the medulla oblongata, descending from the brain stem and enabling parasympathetic control of the lungs, heart and digestive tract. It inhibits oxidative stress, inflammation and sympathetic activity and associated hypoxia. The vagus nerve is commonly referred to in the singular, but is actually a pair of nerves, namely the right and left vagus nerves. The vagus nerve represents the main component of the parasympathetic nervous system, which oversees a vast array of crucial bodily functions, including control of mood, immune response, digestion, and heart rate. It supplies motor parasympathetic fibers to all the organs (except the adrenal glands), from the neck down to the second segment of the transverse colon. The vagus also controls a few skeletal muscles. It establishes one of the connections between the brain and the gastrointestinal tract and sends information about the state of the inner organs to the brain via afferent fibers. By treating the vagus nerve with any wavelength of light from 405-650 nm, alone or in combination, the immune response from the digestive track can be slowed down and reduce symptoms until the patient overcomes ARDS.

To boost immune function the neurological system should also be treated with light therapy. The nerves from the cervical and thoracic spine are the neurological nerves that are tied to the lungs. Spinous process is a bony projection off the posterior of each vertebra. The spinous process protrudes posteriorly from the neural arch and the junction of two laminae and provides the point of attachment for muscles and ligaments of the spine. Applying light energy to the spinous process, from the front of the neck to C3 to C8 vertebrae, especially C5 to C7, or from the back of the patient to T1-T8 vertebrae, will increase neurological function to assist in treating the immune response. Furthermore, light from 400-700 nm has been proven enhance nerve function so any wavelength or combination of wavelengths from 400-700 nm can be implemented in the current invention.

Another pathway that will enhance immune function for ARDS is applying light to the patient's skin at or near the lungs (front and back), vagus nerve, and spinous process having wavelength from 405 nm-650 nm, alone or in combination, which is absorbed by cytochrome c oxidase. Cytochrome c is a terminal enzyme of the electron transport chain and facilitates the transfer of electrons from water-soluble cytochrome c into oxygen, playing an important role in lung function.

Another immune function marker is cytokine interleukin IL-10. IL-10 is important because it has been called the master regulator of the immune system, when IL-10 is boosted it negates the dangerous effects of IL-8 and IL-6. The most effective wavelength reduce IL-10 is 640 nm. This can be enhanced by using other wavelengths such as 405 nm to down-regulate IL-8 which is prevalent when ARDS manifests itself.

To treat a patient with ARDS, light energy 12 is applied externally to the patient 10 in one or more targeted areas of the body. The light energy is applied to the patient's skin at or near the lungs, vagus nerve 7, and spinous process; this application is also referred to in shorthand as applying light energy at or near the lungs, vagus nerve 7, and spinous process. In a preferred embodiment, the targeted areas of the body treated with the light energy are the lungs from both the front (chest) and back of the patient, the vagus nerve from the patient's neck, and the spinous process from C3 to T8 vertebrae from the patient's neck or back. The order that the areas are treated in does not change the effectiveness. In addition, any other additional area of the body may be treated with light energy to enhance immune function.

The light source emits one or more wavelengths in the range of 400 nm to 700 nm, with the desired wavelength within the spread from nominal. Commercial semiconductor laser diodes and other light energy emitters have a spread of ±10 nm from nominal. A person skilled in the art would recognize that “about” means±10 nm from the stated wavelength. For example, if a 635 nm wavelength is specified for treatment, a laser denoted as a 640 nm laser would suffice, because 635 nm is within the spread from nominal. In some embodiments multiple wavelengths are used, either in series, alternately, or simultaneously.

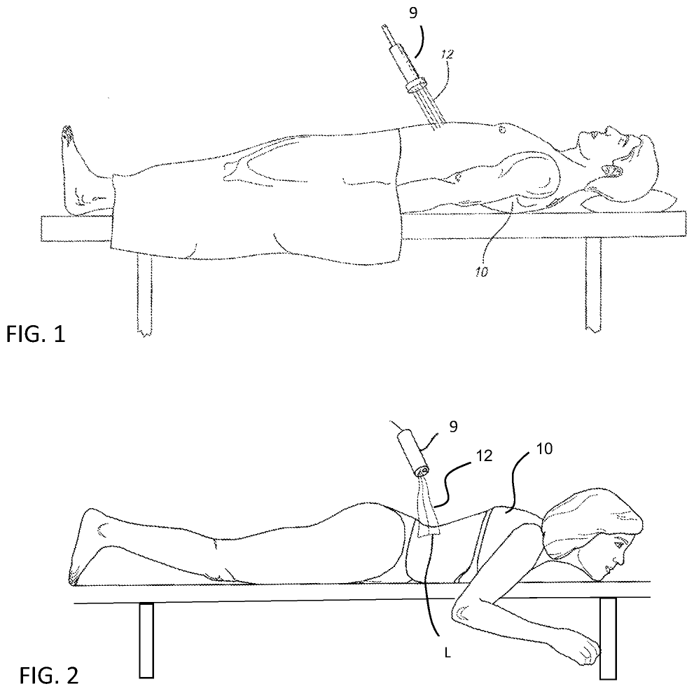

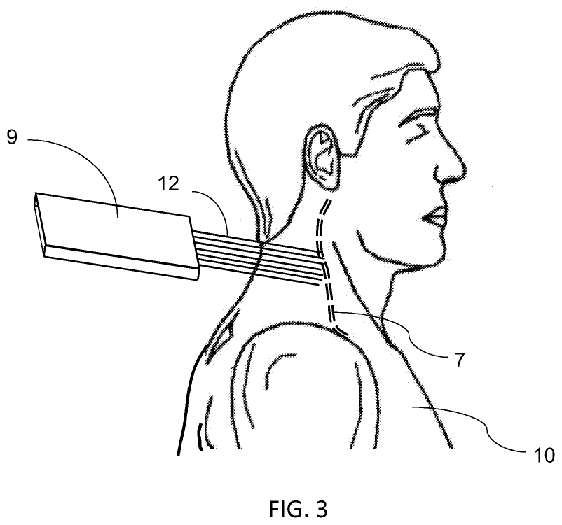

The light can be from any source including light-emitting diodes, hard-wired lasers, or laser diodes, but preferably is from one or more semiconductor laser diodes. The light can be emitted from the same light emitter or from multiple emitters. Preferably the light is emitted as a line from a hand-held laser device, as shown in U.S. Pat. No. 6,746,473 which is incorporated by reference herein, and the line L is waved manually across a person's tissue in the targeted area in a continuous, sweeping manner. FIGS. 1, 2, and 3 illustrate hand-held light emitting devices 9, each containing a laser diode, being used to apply light energy. Alternatively, light can be applied with a scanning device, such as that shown in U.S. Pat. No. 7,947,067 which is incorporated by reference herein. Preferably the power is less than 500 mW. The wavelength, low power, and length of treatment cause no detectable temperature rise of the treated tissue and no macroscopically visible changes in tissue structure.

In some embodiments, the applied light energy is applied with a pulse frequency or frequencies that mimic healthy brain function of alpha (8-12 Hz), beta (13-38 Hz), delta (1-3 Hz), and theta waves (4-7 Hz). The pulse frequencies can be applied singularly, serially, alternately, or simultaneously.

The light energy can be applied to a patient who is lying down, sitting, or standing up.

In a preferred embodiment, a therapeutic amount of light energy at both 405 nm and 635 nm is applied to each area of the body, at the lungs, vagus nerve, and spinous process and optionally any other area, typically for about 15 minutes to each area. The patient is treated 2 times per day until the symptoms start going away, at which time the patent may be treated once per day. Treatments continue until the symptoms are gone, typically in 5 days. The dosages cause no detectable temperature rise of the treated tissue and no macroscopically visible changes in tissue structure.

### Example 1

A patient is diagnosed with ARDS by measuring the patient's blood oxygen level and determining it is low, for example with an oxygen level of less than 92%. Typically the blood oxygen level, also known as oxygen saturation, is determined by pulse oximetry. A device using semiconductor diode lasers emits a combination of 635 nm and 405 nm light, with maximum power of 20 mW total, is used to apply laser light externally to a patient's lungs. The laser energy is applied for about 10 minutes in a back-and-forth sweeping motion across the front of the patient's chest without touching the patient, and the laser energy is applied for about 10 minutes in a back-and-forth sweeping motion externally across the patient's back without touching the patient. A person with skill in the art will understand that “about 10 minutes” means 8-12 minutes. The laser energy is applied so that there is no temperature rise or macroscopically visible change in the targeted areas. Treatment is repeated 2 times a day until the oxygen levels normalize, typically above 95%.

### Example 2

A 635 nm semiconductor diode laser with maximum power of 10 mW is used to apply laser light externally to a patient's tissue at or near the lungs, vagus nerve, gut, and spinous process. The laser energy is applied for 15 minutes to each area in a back-and-forth sweeping motion without touching the patient. The laser light device is programmed with a set of pulse frequencies that include both constant and pulsed wave. This non-invasive procedure produces the same inflammatory reduction seen with corticosteroids, to assist the patient with breathlessness and coughing.

### Example 3

A patient suffering from mild ARDS uses a 635 nm semiconductor diode laser device with maximum power of 20 mW. The laser light energy is applied externally to the patient's tissue at or near the spinous process, from the front of the neck C3 to C8 vertebrae, in a back and forth sweeping motion for 10 minutes without touching the patient. The device is then applied externally to the patient's thorax from the patient's back from T1-T8 for 10 minutes in a back and forth sweeping motion. The device then uses a combination of 635 nm and 405 nm semiconductor laser light, applied externally over the patient's vagus nerve, starting at the patient's neck and following the digestive track to the gut. The laser light is applied in a back-and-forth motion with a maximum power of 20 mW. The laser light will increase neurological function to assist in treating the immune response. Treatment is repeated 2 times a day until the symptoms are reduced and the patient overcomes ARDS.

### Example 4

A patient that had previously experienced symptoms of ARDS uses a device emitting a combination of 635 nm and 405 nm laser light from semiconductor diodes. The lasers use a maximum power of 20 mW laser light total that is applied 10 minutes externally over a patient's lungs. Treatment is repeated 2 times a week over 8 weeks. The laser energy is applied so that there is no temperature rise or macroscopically visible change in the targeted areas. A CAT scan shows the significant improvement in the reduction of fibrous in lungs.

Finally, in addition to the methods set forth above, stem cells may be treated with 532 nm light energy for stem cell de-differentiation. The stem cells are them injected into the patient.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments and equivalents falling within the appended claims.

1. A method for treating acute respiratory distress syndrome in a patient, the method comprising:
a. applying light energy externally to the patient's tissue at targeted areas; wherein
b. the targeted areas comprise:
i. at or near the patient's lungs;
ii. at or near the patient's vagus nerve; and
iii. at or near the patient's spinous process.

2. The method of claim 1 wherein the targeted areas further comprise the patient's gut.

3. The method of claim 1 wherein the light energy is applied at one or more wavelengths in the range of 400 nm-700 nm.

4. The method of claim 1 wherein the light energy is applied at 405 nm and 635 nm.

5. The method of claim 1 wherein the applied light energy causes no detectable temperature rise of the treated tissue.

6. The method of claim 1 wherein the light energy is emitted from a laser.

7. The method of claim 1 wherein the light energy is applied with a handheld laser device in a sweeping motion without touching the patient.

8. A method for treating acute respiratory distress syndrome in a patient, the method comprising:
a. applying light energy externally to:
i. the patient's chest or back;
ii. the patient's neck; and
iii. the patient's C3-C8 vertebrae.

9. The method of claim 8 wherein the targeted areas further comprise the patient's gut.

10. The method of claim 8 wherein the light energy is applied at one or more wavelengths in the range of 400 nm-700 nm.

11. The method of claim 8 wherein the light energy is applied at 405 nm and 635 nm.

12. The method of claim 8 wherein the applied light energy causes no detectable temperature rise of the treated tissue.

13. The method of claim 8 wherein the light energy is emitted from a laser.

14. The method of claim 8 wherein the light energy is applied with a handheld laser device in a sweeping motion without touching the patient.

15. A method for treating acute respiratory distress syndrome in a patient, the method comprising:
a. measuring the patient's blood oxygen level;
b. if the patient's blood oxygen level is less than 92%, applying light energy externally to the patient's chest or back;
c. measuring the patient's blood oxygen level;
d. if the patient's blood oxygen level is less than 95%, again applying light energy externally to the patient's chest or back.

16. The method of claim 15 wherein the light energy is applied for about 10 minutes.

17. The method of claim 15 wherein the light energy is applied at one or more wavelengths in the range of 400 nm-700 nm.

18. The method of claim 15 wherein the light energy is applied at 405 nm and 635 nm.

19. The method of claim 15 wherein the applied light energy causes no detectable temperature rise of the treated tissue.

20. The method of claim 15 wherein the light energy is applied with a handheld laser device in a sweeping motion without touching the patient.
